(19) United States
(12) Patent Application Publication
(10) Pub. No.: US 2026/0137705 A1
(43) Pub. Date: May 21, 2026

(54) NOVEL CANNABINOID NANOPARTICLE COMPOSITIONS FOR THERAPEUTIC APPLICATIONS IN VETERINARY MEDICINE

(71) Applicant: Chou2 Pharma LLC, Narberth, PA (US)

(72) Inventors: Stuart Wilcox, Woodstock, GA (US); Blake Smith, Salt Lake City, UT (US)

(21) Appl. No.: 19/393,372

(22) Filed: Nov. 18, 2025

Related U.S. Application Data
(60) Provisional application No. 63/722,025, filed on Nov. 18, 2024.
(60) Provisional application No. 63/722,038, filed on Nov. 18, 2024.
(60) Provisional application No. 63/722,049, filed on Nov. 18, 2024.

(51) Int. Cl. A61K 31/00; A61K 9/10; A61K 9/14; A61K 47/14; A61K 47/44; A61P 25/00; A61P 25/08; A61P 25/22; B82Y 5/00

(52) U.S. Cl. CPC A61K 31/658; A61K 9/10; A61K 9/14; A61K 47/14; A61K 47/44; A61P 25/00; A61P 25/08; A61P 25/22; B82Y 5/00

(57) ABSTRACT

The present invention relates to methods and pharmaceutical compositions for treating conditions in non-human mammals, including seizures, idiopathic epilepsy, anxiety, noise aversion, and separation anxiety. The treatment involves administering cannabinoid compositions comprising at least one cannabinoid selected from cannabidiol (CBD), cannabidivarin (CBDV), cannabichromene (CBC), cannabigerol (CBG), cannabinol (CBN), and tetrahydrocannabivarin (THCV). Specific embodiments comprise CBD isolate nano-particles with purity greater than 99% of total cannabinoids and particle sizes less than 100 nanometers, suspended in oil-based media, essentially free of tetrahydrocannabinol (THC). Methods include acute treatment for noise aversion administered immediately prior to or during noise events, and daily administration for separation anxiety management. A pharmaceutical composition comprising CBD, CBDV, and CBC in a 5:1:1 weight ratio for treating seizures and idiopathic epilepsy is disclosed. The compositions may be administered orally, sublingually, or topically at dosages of 0.1 to 10 mg per kg body weight in cats, dogs, and horses.

### CROSS-REFERENCE TO RELATED APPLICATIONS

This application claims the benefit of priority to:

U.S. Provisional Patent Application No. 63/722,025, filed Nov. 18, 2024, titled “NOVEL CANNABINOID NANOPARTICLE COMPOSITIONS FOR THERAPEUTIC APPLICATIONS IN VETERINARY MEDICINE”;

U.S. Provisional Patent Application No. 63/722,038, filed Nov. 18, 2024, titled “NOVEL CANNABINOID COMPOSITIONS FOR TREATING STRESS AND ANXIETY INDUCED BY NOISE IN VETERINARY MEDICINE”; and

U.S. Provisional Patent Application No. 63/722,049, filed Nov. 18, 2024, titled “NOVEL CANNABINOID COMPOSITIONS FOR TREATING SEPARATION ANXIETY IN VETERINARY MEDICINE”.

The entire contents of each of the above applications are incorporated herein by reference.

### FIELD OF THE INVENTION

The present invention relates to the field of THC-free cannabinoid-based therapeutics. Specifically, it pertains to the use of non-psychoactive cannabinoids, such as cannabidiol (CBD) and other cannabinoid derivatives that are free of tetrahydrocannabinol (THC), in the treatment and management of various diseases and disorders in veterinary medicine. The invention is directed toward the treatment of animals such as cats, dogs, and horses. Conditions treated include, but are not limited to, seizures, idiopathic epilepsy, anxiety, noise aversion, separation anxiety, appetite stimulation, appetite control, anti-inflammatory conditions, anti-allergy responses, and various dermatological conditions, such as pruritus (itching). The invention encompasses formulations, delivery systems, and methods for administering THC-free cannabinoids for therapeutic use in these animals without the psychoactive effects of THC.

### BACKGROUND OF THE INVENTION

Cannabinoids are increasingly gaining recognition for their remarkable medicinal effects in veterinary medicine, particularly in the treatment of cats, dogs, and horses. Among these compounds, there are approximately 120 different cannabinoids that naturally occur in various Cannabis plant species. These compounds have numerous potential therapeutic benefits, including seizure management, idiopathic epilepsy, anxiety and stress reduction, noise aversion, separation anxiety, appetite stimulation and control, anti-inflammatory properties, anti-allergy responses, and pruritus management. Consequently, cannabinoids present an exciting opportunity as drug candidates for the treatment of a diverse range of conditions in veterinary applications.

Cannabinoids intended for therapeutic use in animals can be sourced from industrial hemp, which includes various strains of Cannabis sativa that have low concentrations of THC but contain a range of other cannabinoids. A significant advantage of industrial hemp is its high content of cannabidiol (CBD), as well as other non-psychoactive cannabinoids such as cannabidivarin (CBDV), cannabichromene (CBC), cannabigerol (CBG), cannabinol (CBN), and cannabitriol (CBT), with only trace amounts of tetrahydrocannabinol (THC), the primary psychoactive compound in Cannabis. This unique cannabinoid profile positions hemp as the preferred source for THC-free therapeutic formulations in animals, ensuring that treatment is effective without the psychoactive effects associated with THC-containing Cannabis.

The compositions of hemp extract are intricate, containing a mix of cannabinoids, terpenes, and other natural compounds, which can offer a broad range of potential health benefits. Full spectrum cannabinoids refer to the complete array of cannabinoids, terpenes, flavonoids, and other natural compounds found in the cannabis plant, preserved together in a product. Unlike isolates, which contain only one cannabinoid (e.g., THC or CBD), full-spectrum products retain a wide range of cannabinoids (such as THC, CBD, CBG, CBN, and others) as well as non-cannabinoid compounds like terpenes and flavonoids. The combination of these compounds is thought to produce an “entourage effect,” where the compounds work synergistically to enhance each other's therapeutic effects. However, full-spectrum products are naturally derived and the composition and purity vary with each extraction and preparation.

The complex interactions between these compounds can make it challenging to predict pharmacological effects reliably in veterinary patients. This complexity underscores the importance of pure isolates, such as CBD and CBG isolates, which provide a refined and controlled form of these cannabinoids. By isolating individual cannabinoids from hemp extract, precise dosages can be calibrated for veterinary therapeutic use, ensuring a consistent and reliable therapeutic outcome without the variability seen in hemp extract compositions. These pure cannabinoid isolates offer a more dependable and tailored approach to treating conditions in cats, dogs, and horses, providing a significant step forward in veterinary cannabinoid-based therapies.

Verrico et al. conducted a randomized, double-blind, placebo-controlled study to evaluate the efficacy of daily cannabidiol (CBD) for managing osteoarthritis pain in dogs. The trial involved 20 dogs diagnosed with osteoarthritis, who were randomly assigned to receive either a CBD formulation or a placebo for four weeks. Pain was assessed using a validated scale, and physical function was evaluated through activity measurements. Results indicated that dogs treated with CBD experienced a significant reduction in pain and improved function compared to those receiving the placebo, with no notable adverse effects observed, suggesting that CBD may be a promising therapeutic option for canine osteoarthritis pain management (Verrico C D, Wesson S, Konduri V, et al. A randomized, double-blind, placebo-controlled study of daily cannabidiol for the treatment of canine osteoarthritis pain. Pain. 2020 September; 161 (9): 2191-2202).

Brioschi et al. conducted a study evaluating the effects of an oral transmucosal cannabidiol (CBD) oil formulation as part of a multimodal analgesic regimen for dogs suffering from spontaneous osteoarthritis. The study aimed to assess CBD's impact on pain relief and quality of life improvement. Results showed that the dogs receiving CBD experienced significant reductions in pain and discomfort, as well as improved mobility and overall quality of life. The authors concluded that CBD, when used as part of a broader pain management strategy, can be an effective adjunct in treating osteoarthritis-related pain in dogs, warranting further clinical exploration. (Brioschi, F. A.; Di Cesare, F.; Gioeni, D.; Rabbogliatti, V.; Ferrari, F.; D′Urso, E. S.; Amari, M.; Ravasio, G. Oral Transmucosal Cannabidiol Oil Formulation as Part of a Multimodal Analgesic Regimen: Effects on Pain Relief and Quality of Life Improvement in Dogs Affected by Spontaneous Osteoarthritis. Animals 2020, 10, 1505.)

Khodadadi et al. investigated the effects of cannabidiol (CBD) on cognitive function in a murine model of Alzheimer's disease. The study highlighted that CBD administration led to significant improvements in cognitive performance, attributed to its regulatory effects on interleukin-33 (IL-33) and the upregulation of triggering receptor expressed on myeloid cells 2 (TREM2). These molecular changes are thought to enhance neuroinflammation and support the brain's immune response, potentially mitigating the neurodegenerative processes associated with Alzheimer's. The research focused on the effects of CBD on amyloid-beta plaques, which are associated with Alzheimer's pathology. The findings indicated that CBD treatment led to a significant reduction in plaque buildup and improved performance in cognitive tests, suggesting its potential as a therapeutic agent for Alzheimer's disease. The authors emphasized that these results support further investigation into CBD's effects on neurodegenerative conditions and highlight its possible role in mitigating Alzheimer's-related symptoms. (CBD reduces plaque, improves cognition in model of familial Alzheimer's.” ScienceDaily. ScienceDaily, 9 Mar. 2021).

Inkol, Hocker, and Mutsaers explored the effects of combining cannabidiol (CBD) with chemotherapeutic agents in treating canine urothelial carcinoma cells. The researchers found that CBD enhanced the anti-cancer effects of certain chemotherapeutic drugs, improving their ability to inhibit cancer cell growth. CBD appeared to increase the sensitivity of cancer cells to these therapies, suggesting a potential synergistic effect. While the results were promising in vitro, the study emphasizes the need for further investigation into the safety and efficacy of CBD as part of combination therapy for canine cancers in clinical settings. (Inkol J M, Hocker S E, Mutsaers A J (2021) Combination therapy with cannabidiol and chemotherapeutics in canine urothelial carcinoma cells. PLOS ONE 16 (8)).

Bialer and Perucca examined whether cannabidiol (CBD) has independent antiseizure effects or if its efficacy is primarily due to interactions with clobazam, a commonly used anticonvulsant. The authors analyzed evidence from randomized controlled trials, particularly focusing on studies where CBD was used alongside clobazam. While the data indicated that CBD can significantly reduce seizure frequency, the authors noted that much of this effect could be attributed to CBD's interaction with clobazam, which increases clobazam's levels in the body. However, there is also evidence suggesting that CBD may have its own antiseizure properties, independent of clobazam. The authors concluded that while CBD likely has inherent antiseizure activity, further studies are needed to fully clarify the extent of its independent efficacy and the potential contribution of drug interactions. (Bialer M, Perucca E. Does cannabidiol have antiseizure activity independent of its interactions with clobazam? An appraisal of the evidence from randomized controlled trials. Epilepsia. 2020; 61:1082-1089).

Fear, anxiety, and stress affect a significant portion of companion animals, with veterinarians estimating that over half of dogs and cats experience these issues. Anxiety in animals is a complex condition that can arise from various sources and manifest in multiple forms. Fear-related anxiety can be triggered by loud noises, unfamiliar people or animals, visual stimuli such as hats or umbrellas, new or strange environments, specific situations like veterinary visits or car rides, or even certain surfaces like grass or wood floors. While some animals may experience brief reactions to these stimuli, those with underlying anxiety disorders may have more significant, lasting responses that impact their well-being.

Separation anxiety (SA) is another prevalent condition that affects many companion animals. It is estimated to impact around 14 percent of dogs, with similar behaviors observed in cats and, to some extent, in horses. Unlike simple behavioral issues, separation anxiety is characterized by profound distress when an animal is left alone or separated from its primary caregiver. Animals with SA often display destructive behaviors, such as urinating or defecating indoors, chewing furniture, or vocalizing excessively. This is a serious condition that reflects an inability to self-soothe or find comfort when alone.

Noise aversion is particularly prevalent among dogs, with research indicating that nearly 70% of dogs in the United States exhibit one or more signs of noise-related anxiety. While these problems are often misunderstood as purely behavioral, they are increasingly recognized as medical conditions. Chronic exposure to anxiety-inducing stimuli can harm a dog's physical, mental, and social health, ultimately diminishing quality of life.

Cannabidiol (CBD), is a non-psychoactive compound. It was shown promise in human studies for its anxiolytic effects. For example, single-dose studies have demonstrated CBD's effectiveness in reducing anxiety during simulated public speaking tasks at doses of 300 mg to 600 mg. In animal studies, lower doses, such as 10 mg/kg in rats, have shown a stronger anxiolytic effect than higher doses, suggesting potential therapeutic windows. Anecdotal evidence exists regarding the use of over-the-counter hemp-derived CBD to manage anxiety in pets, but no formal studies or published data currently support CBD therapy for veterinary anxiety treatment.

While dosage guidelines for dogs are limited, recommendations for CBD use range from 0.1 to 0.5 mg/kg of body weight per day. Over the past forty years, numerous drugs have been developed to treat anxiety in animals, such as Xanax, and in 2016, the FDA approved Sileo specifically for noise aversion in dogs. However, no FDA-approved veterinary drugs to date incorporate cannabinoids, underscoring a gap in research and development for natural compound therapies in treating anxiety and noise aversion in pets.

Greb and Puschner explore various conditions where cannabinoid treats might be used as adjunctive therapy in pets. These include chronic pain management, such as that associated with osteoarthritis or neuropathic pain, where cannabinoids may offer relief. Anxiety disorders, like separation anxiety or reactions to loud noises, are another area where cannabinoids could help pets. They also mention the potential of cannabinoids to stimulate appetite in animals suffering from conditions that diminish their desire to eat. Additionally, cannabinoids may have anticonvulsant properties, making them a possible therapy for pets with epilepsy. Inflammatory conditions, such as inflammatory bowel disease or skin issues like dermatitis, could also benefit from cannabinoid therapy due to its anti-inflammatory effects. Furthermore, cannabinoids may help alleviate cancer-related symptoms, including pain, nausea, and loss of appetite. Greb and Puschner stress that while these applications are promising, more research is essential to understand proper dosing, safety, and effectiveness for cannabinoid use in veterinary care. (Alexandra Greb & Birgit Puschner (2018) Cannabinoid treats as adjunctive therapy for pets: gaps in our knowledge, Toxicology Communications, 2:1, 10-14).

The following sections provide a detailed description of the proposed THC-free cannabinoid formulations, including their key features, benefits, and potential applications in veterinary medicine. The invention offers veterinarians and animal owners a reliable and effective option for managing a wide array of conditions, enhancing the overall well-being of animals while eliminating the psychoactive effects associated with THC.

### SUMMARY OF THE INVENTION

The present invention provides novel cannabinoid-based pharmaceutical compositions and methods for treating various conditions in non-human mammals, particularly cats, dogs, and horses. The invention addresses the need for effective, non-psychoactive therapeutic options for managing neurological, behavioral, and inflammatory conditions in veterinary medicine.

In one aspect, the invention provides a method for treating a condition in a non-human mammal by administering a therapeutically effective amount of a cannabinoid composition comprising at least one cannabinoid selected from cannabidiol (CBD), cannabidivarin (CBDV), cannabichromene (CBC), cannabigerol (CBG), cannabinol (CBN), cannabinodiol (CBDL), cannabielsoin (CBE), cannabitriol (CBT), cannabicyclol (CBL), and tetrahydrocannabivarin (THCV). Conditions treated include seizures, idiopathic epilepsy, anxiety, noise aversion, separation anxiety, appetite stimulation, appetite control, anti-inflammation, anti-allergy, pruritus, and dermatological disorders. The cannabinoid composition may be administered orally, sublingually, or topically in various forms including liquids, oils, gels, tablets, chews, lotions, and creams. The therapeutically effective amount typically ranges from 1 mg/kg to 10 mg/kg of body weight per day.

In another aspect, the invention provides a veterinarian medicine composition comprising cannabidiol (CBD) isolate nano-particles suspended in an oil-based medium, wherein the CBD isolate has a purity of greater than 99% of total cannabinoids and an average particle size less than 100 nanometers, preferably less than 80 nanometers, and more preferably between 20 and 60 nanometers. The composition is essentially free of tetrahydrocannabinol (THC), typically containing less than 0.1% THC, ensuring non-psychoactive therapeutic effects. The nano-particle formulation provides enhanced bioavailability, rapid absorption, and consistent dosing compared to traditional cannabinoid formulations. The oil-based medium may be selected from medium-chain triglyceride (MCT) oil, hemp seed oil, olive oil, sunflower oil, grapeseed oil, fish oil, and avocado oil, with MCT oil being particularly preferred.

The veterinarian medicine composition may further comprise additional cannabinoid isolate nano-particles, including cannabichromene (CBC) isolate nano-particles and/or cannabigerol (CBG) isolate nano-particles, each with particle sizes less than 100 nanometers. These multi-cannabinoid formulations allow for synergistic therapeutic effects while maintaining precise control over the ratio and dosage of each cannabinoid component.

In a further aspect, the invention provides a method for treating noise aversion in a non-human mammal by administering an effective amount of a veterinarian medicine composition comprising CBD isolate nano-particles immediately prior to or during a stress or anxiety-inducing noise event. The CBD isolate has a purity greater than 99% of total cannabinoids and particle size less than 100 nanometers, and the composition is essentially free of THC. The method is particularly advantageous for acute management of noise-related anxiety, with administration timing ranging from 60 minutes prior to the noise event to after the onset of the event. Preferred administration is less than 20 minutes prior to the noise event. The administration may be discontinued when the noise event subsides or diminishes to a non-anxiety-inducing level. Noise events include fireworks, thunderstorms, gunfire, construction noise, and airplane flyovers. The dosage typically ranges from 0.1 to 10 mg per kg of body weight, preferably 0.5 to 2 mg per kg of body weight.

In yet another aspect, the invention provides a method for treating separation anxiety in a non-human mammal by administering an effective amount of a veterinarian medicine composition comprising CBD isolate nano-particles suspended in an oil-based medium. The CBD isolate has a purity greater than 99% of total cannabinoids and particle size less than 100 nanometers, and the composition is essentially free of THC. Unlike the acute treatment for noise aversion, the separation anxiety method typically involves daily administration to provide consistent therapeutic management of chronic anxiety symptoms. The dosage ranges from 0.1 to 10 mg per kg of body weight, preferably 0.5 to 2 mg per kg of body weight. The composition may be administered orally or sublingually.

In still another aspect, the invention provides a pharmaceutical composition comprising cannabidiol (CBD) isolate, cannabidivarin (CBDV) isolate, and cannabichromene (CBC) isolate in a weight ratio of 5:1:1, along with a pharmaceutically acceptable carrier, for the treatment of seizures and idiopathic epilepsy in non-human mammals. This specific ratio has been optimized for anticonvulsant efficacy. The cannabinoid isolates may be in nano-particle form suspended in an oil-based medium, with particle sizes less than 100 nanometers, preferably less than 80 nanometers, and more preferably between 20 and 60 nanometers. Each cannabinoid isolate preferably has a purity of at least 99% of total cannabinoids, and the composition is essentially free of THC, containing less than 0.1% THC. The composition may be administered in various forms including orally dosed liquids, oils, gels, tablets, and chews, as well as topically applied lotions and gels.

The invention offers several significant advantages over existing treatments. The use of highly purified cannabinoid isolates ensures consistent and predictable therapeutic outcomes, avoiding the variability associated with full-spectrum or broad-spectrum hemp extracts. The nano-particle formulation dramatically increases bioavailability through enhanced surface area and improved absorption across biological membranes. The nano-sized particles allow for efficient uptake through the oral mucosa and esophagus, effectively bypassing the upper gastrointestinal tract and minimizing degradation by digestive enzymes and stomach acids. This results in faster onset of therapeutic effects and higher concentrations reaching systemic circulation intact. The THC-free formulations eliminate concerns about psychoactive effects, making the treatments safe and appropriate for veterinary applications.

The invention further encompasses methods of combining the cannabinoid compositions with additional therapeutic agents selected based on the condition being treated, including anti-seizure medications, anti-anxiety medications, appetite stimulants, anti-inflammatory medications, anti-allergy medications, and anti-pruritic agents.

These and other aspects, features, and advantages of the present invention will become more fully apparent from the following detailed description and appended claims.

### BRIEF DESCRIPTION OF DRAWINGS

The foregoing summary, as well as the following detailed description of the preferred embodiments, will be better understood when read in conjunction with the appended drawings. It should be understood, however, that the invention is not limited to the precise arrangements and instrumentalities shown. In the drawings,

FIG. 1: Illustrates main molecular targets and potential mechanisms of action of CBD. CBD inhibits both FAAH, the enzyme which metabolizes anandamide, and FABPs, which mediate the transport of anandamide to FAAH; both mechanisms ultimately result in the indirect activation of CB1 and/or CB2 receptors. CBD also activates the 5-HT1A receptor, PPARγ and the transient receptor potential channels TRPV1, TRPA1 and TRPV2. Finally, CBD inhibits adenosine reuptake and antagonizes GPR55, TRPM8 and T-type Ca2+ channels. 5-HT1A and (indirect) cannabinoid receptor activation are the mechanisms that have been implicated in the anxiolytic effects of CBD to date.

### DETAILED DESCRIPTION OF THE INVENTION

The present invention provides a formulation comprising nano-particles of cannabinoids isolates uniformly dispersed in an oil-based medium.

The cannabinoid isolates used in this invention may include, but are not limited to, cannabidiol (CBD), cannabidivarin (CBDV), cannabichromene (CBC), cannabigerol (CBG), cannabinol (CBN), cannabinodiol (CBDL), cannabielsoin (CBE), cannabitriol (CBT), cannabicyclol (CBL), and tetrahydrocannabivarin (THCV). Each of the cannabinoid isolate is essentially pure, having at 99%, or 99.9%, or 99.99% of the total cannabinoids. Total cannabinoids refer to the combined concentration of all cannabinoid compounds present. The cannabinoid isolate having less than 1%, or less than 0.1%, 0.01%, or 0.001% of tetrahydrocannabinol (THC) or other cannabinoids, or contains trace amounts of THC or other cannabinoids, below detectable limits. The formulation that includes any of the cannabinoid isolates is essentially free other cannabinoids, especially THC. Formulation typically contains less than 0.1% of other cannabinoids, or may contain less than 0.01% other cannabinoids, or may contain less than 0.001% other cannabinoids.

Reducing the particle size to the nanoscale significantly increases the surface area of the cannabinoid isolates, which in turn enhances their solubility and facilitates enhanced absorption across biological membranes. This increase in bioavailability allows for improved therapeutic efficacy, ensuring that smaller doses of the nano-particles achieve a comparable or greater effect than traditional non-nano formulations. The nano-particles of cannabinoids isolates are dispersed uniformly within an oil medium, creating a stable formulation that prevents aggregation and ensures that the particles remain evenly distributed over time. This stability enables consistent dosing in each administration and minimizes the variability in cannabinoid isolates delivery to the body. The uniformity and stability of this nano-dispersion are particularly advantageous for maintaining the bioactivity of cannabinoids in various applications, including oral, topical, and transdermal formulations.

The nano-particles of cannabinoids isolates described in this invention offer the advantage of ease of absorption and enhanced bioavailability, with the added benefit of rapid absorption when administered to an animal. When administered orally, the nano-sized particles allow for efficient uptake through the oral mucosa and esophagus, effectively bypassing the upper gastrointestinal (GI) tract. This delivery route minimizes degradation of cannabinoids that would otherwise occur due to exposure to digestive enzymes and stomach acids, preserving the integrity and potency of the cannabinoids.

By avoiding the initial stages of the GI tract, the nano-particle dispersion enables cannabinoids isolates to enter the bloodstream more quickly, providing a faster onset of therapeutic effects. This mechanism not only maximizes the bioavailability of cannabinoids but also ensures that higher concentrations reach systemic circulation intact, enhancing the overall efficacy of the formulation for medical and veterinary applications. The formulation is therefore optimized for both rapid action and sustained potency, making it an ideal solution for therapies targeting the central nervous system (CNS).

The cannabinoids isolates are processed into nano-sized particles with a typical diameter of less than 200 nanometers, or less than 100 nanometers, preferably less than 80 nanometers, more preferably less than 60 nanometers. In one embodiment, the cannabinoids isolates are processed into nano-sized particles with a mean particle size of 10-200 nanometers, 10-100 nanometers, 20-80 nanometers, or 20-60 nanometers. In another embodiment, the cannabinoids isolates are processed into nano-sized particles with a mean particle size of about 40-50 nanometers. The particle size distribution of the cannabinoids isolates is typically less than +50 nanometers, or less than +25 nanometers, or less than +10 nanometers.

Essentially pure cannabinoid isolates (e.g., >99%, or >99.9, or >99.99%), such as cannabidiol (CBD) or other isolated cannabinoids, typically exist in a solid crystalline or powder form at room temperature when isolated to high purity levels. In their pure form, cannabinoid isolates have a melting point above room temperature, which causes them to remain in a stable solid state under standard conditions. When essentially pure cannabinoid isolates are suspended in an oil-based medium without further processing, the particle size typically remains in the range of hundreds of nanometers, often greater than 300 nanometers. At this scale, the cannabinoids may not achieve optimal bioavailability or stability, as larger particles are less readily absorbed and can settle or aggregate within the medium over time. To enhance their therapeutic effectiveness, the cannabinoid particles must be carefully processed into nano-particle size, typically less than 100 nanometers.

The cannabinoids isolates are processed into nano-sized particles through either mechanical or chemical processes. Mechanical methods may include high-shear mixing, high-pressure homogenization, ultrasonication, milling, or in a fluidized bed reactor, which reduce particle size by applying physical forces to break down the cannabinoids into nanoscale dimensions. Chemical processes may involve solvent evaporation, precipitation, or emulsification techniques that use chemical agents or stabilizers to achieve and maintain the desired particle size. These methods ensure that the cannabinoids reach a uniform nano-sized state, allowing for enhanced stability and bioavailability in the final formulation.

In high-shear mixing, the oil and cannabinoid isolate mixture is subjected to intense shear forces, which break down the cannabinoid particles to sizes below 100 nanometers. In high-pressure homogenization, a cannabinoid isolates mixture is forced through a narrow nozzle under high pressure, generating intense shear forces and turbulence that break down the particles into nano-sized dimensions. Ultrasonication employs high-frequency sound waves to create microscopic cavitation bubbles within the cannabinoid solution. As these bubbles collapse, they release energy that disrupts and reduces the particles to nano-scale, creating a fine and stable dispersion. The milling process involves grinding solid cannabinoid particles into smaller sizes through mechanical action, often using beads or similar media. This process is typically performed in a liquid environment to prevent aggregation, producing a uniform nano-sized particle dispersion.

Using a fluidized bed reactor (FBR) to produce cannabinoid isolate nano-particles involves suspending cannabinoid precursor materials in the reactor as a gas or liquid flows upward, creating a fluidized state that promotes controlled reaction conditions and precise particle formation. In this setup, cannabinoid precursors are introduced into the fluidized bed, where they undergo rapid nucleation and are reduced to nano-sized particles under carefully regulated temperature, pressure, and concentration conditions.

Solvent Evaporation: In solvent evaporation, cannabinoids isolates are first dissolved in a volatile solvent. Upon evaporation of the solvent, the cannabinoids precipitate as nano-sized particles. Stabilizers may be incorporated to maintain the nano-scale and prevent aggregation. The precipitation method involves dissolving cannabinoids isolates in a solvent, followed by the introduction of a non-solvent, leading to the precipitation of cannabinoids as nano-sized particles. Stabilizing agents are commonly used to preserve particle size and avoid clumping. Emulsification involves dispersing cannabinoids within an oil or aqueous phase using surfactants to form a nano-emulsion. Mechanical forces, such as high-speed stirring, are applied to further reduce particle size, resulting in stable nano-sized particles suitable for enhanced bioavailability.

Suitable oil-based medium for dispersing and suspending cannabinoid isolates include several options that enhance stability, bioavailability, and therapeutic efficacy. Medium-chain triglyceride (MCT) oil, is a popular choice due to its high absorption rate and neutral flavor, which make it an ideal base for nano-dispersion of cannabinoid isolates. Another effective option is hemp seed oil, which is naturally compatible with cannabinoids and contains beneficial omega-3 and omega-6 fatty acids, creating a smooth, stable medium for oral formulations. Olive oil is also widely used for its antioxidant properties and health benefits, offering a stable lipid-rich medium that enhances cannabinoid absorption, especially in nutraceutical and pharmaceutical applications. Sunflower oil, high in linoleic acid, provides a stable, mild-tasting carrier suitable for both topical and oral products. It offers a smooth consistency that can enhance the user experience in edible formulations. Avocado oil supplies monounsaturated fats, providing a stable and smooth medium ideal for both edible and topical applications. Similarly, grapeseed oil, with its light texture and fast absorption rate, is especially useful in topical formulations where quick skin penetration is desired without a greasy residue. Each of these oils offers unique properties to optimize cannabinoid suspension and delivery across a variety of formulations. Other oil-based medium such as fish oil are also contemplated to be used with the cannabinoids isolates formulation.

In one embodiment, the formulation comprises cannabinoid isolates suspended in an oil-based medium that are mechanically processed to achieve nano-particle size. This formulation begins with CBD isolate (or other cannabinoids) combined with a suitable carrier oil, such as medium-chain triglyceride (MCT) oil. The mixture is processed using either high-pressure homogenization or ultrasonication to reduce the particle size of the cannabinoids to below 100 nanometers, or below 80 nanometers, preferably between 20 and 60 nanometers.

Particle size of cannabinoid isolates in an oil-based medium can be analyzed using several analytical techniques. These methods provide precise measurements of particle size distribution and help ensure that the formulation meets the target nano-particle specifications. Common techniques include Dynamic Light Scattering (DLS), Transmission Electron Microscopy (TEM), Scanning Electron Microscopy (SEM), and Laser Diffraction.

DLS is a widely used, non-invasive method for measuring nano-particle size in a liquid suspension. In this technique, a laser is directed at the sample, and light scattered by the particles is analyzed to determine their size based on Brownian motion. DLS is effective for measuring particle sizes in the range of 1 nm to 1 micron and provides a fast, accurate way to measure nano-particle size in oil-based cannabinoid formulations.

TEM offers high-resolution imaging that allows for direct visualization of particle size and morphology. In this technique, a beam of electrons is transmitted through an ultrathin sample of the cannabinoid-oil suspension, producing a detailed image that can reveal particle dimensions at the nano scale. TEM is highly accurate for particle size measurements.

SEM provides detailed images of particle surface morphology and size. In this method, an electron beam scans the surface of the sample, producing high-resolution images. Although SEM typically requires sample drying and preparation, it is useful for confirming particle size and distribution in oil-based nano-particle suspensions.

Laser diffraction measures particle size by analyzing the scattering pattern of light as it passes through the suspension. This method is effective for a wide range of particle sizes, from sub-micron to millimeter scales, and is commonly used for rapid particle size analysis in emulsions and suspensions. Laser diffraction is valuable for assessing the overall size distribution within cannabinoid nano-dispersions.

Exemplary cannabinoid isolates formulations:

CBD Oral Tincture: 300 mg of CBD isolate, 30 ml of MCT oil. Dissolve CBD isolate into MCT oil mechanically suspend and reduce particle size until CBD is fully suspended and the particle size is between 20-80 nanometers, creating a uniform tincture. This formulation provides a convenient dosing option with high bioavailability.

CBD Oral Tincture: 500 mg of CBD isolate, 30 ml of MCT oil. Dissolve CBD isolate into MCT oil mechanically suspend and reduce particle size until CBD is fully suspended and the particle size is between 20-80 nanometers, creating a uniform tincture. This formulation provides a convenient dosing option with high bioavailability.

CBD/CBG (1:2) formulation in MCT oil: 500 mg of CBD isolate, 1000 mg of CBG isolate, and 30 ml of MCT oil. Measure 500 mg of CBD isolate and 1000 mg of CBG isolate to achieve a 1:2 ratio of CBD to CBG. Add the CBD and CBG isolates to 30 ml of MCT oil in a mixing container. Mechanically suspend and reduce particle size until CBD and CBG is fully suspended and the particle size is between 20-80 nanometers, creating a uniform tincture. This formulation provides a balanced 1:2 ratio of CBD to CBG in an MCT oil base, ideal for applications where both cannabinoids are desired in a specific therapeutic ratio. This formulation can be used as an oral tincture, soft gel filling, or in other delivery forms where precise ratios of cannabinoids are preferred.

CBD/CBG (1:1) formulation in MCT oil: 250 mg of CBD isolate, 250 mg of CBG isolate, and 30 ml of MCT oil. Measure 250 mg of CBD isolate and 250 mg of CBG isolate to achieve a 1:1 ratio of CBD to CBG. Add the CBD and CBG isolates to 30 ml of MCT oil in a mixing container. Mechanically suspend and reduce particle size until CBD and CBG is fully suspended and the particle size is between 20-80 nanometers, creating a uniform tincture. This formulation provides a balanced 1:1 ratio of CBD to CBG in an MCT oil base, ideal for applications where both cannabinoids are desired in a specific therapeutic ratio. This formulation can be used as an oral tincture, soft gel filling, or in other delivery forms where precise ratios of cannabinoids are preferred.

CBD/CBG (3:1) formulation in MCT oil: 300 mg of CBD isolate, 100 mg of CBG isolate, and 30 ml of MCT oil. Measure 300 mg of CBD isolate and 100 mg of CBG isolate to achieve a 3:1 ratio of CBD to CBG. Add the CBD and CBG isolates to 30 ml of MCT oil in a mixing container. Mechanically suspend and reduce particle size until CBD and CBG is fully suspended and the particle size is between 20-80 nanometers, creating a uniform tincture. This formulation provides a balanced 3:1 ratio of CBD to CBG in an MCT oil base, ideal for applications where both cannabinoids are desired in a specific therapeutic ratio. This formulation can be used as an oral tincture, soft gel filling, or in other delivery forms where precise ratios of cannabinoids are preferred.

CBD/CBG (9:1) formulation in MCT oil: 450 mg of CBD isolate, 50 mg of CBG isolate, and 30 ml of MCT oil. Measure 450 mg of CBD isolate and 50 mg of CBG isolate to achieve a 9:1 ratio of CBD to CBG. Add the CBD and CBG isolates to 30 ml of MCT oil in a mixing container. Mechanically suspend and reduce particle size until CBD and CBG is fully suspended and the particle size is between 20-80 nanometers, creating a uniform tincture. This formulation provides a balanced 9:1 ratio of CBD to CBG in an MCT oil base, ideal for applications where both cannabinoids are desired in a specific therapeutic ratio. This formulation can be used as an oral tincture, soft gel filling, or in other delivery forms where precise ratios of cannabinoids are preferred.

CBD:CBC:CBG (8:1:1) formulation in MCT oil: 400 mg of CBD isolate, 50 mg of CBC isolate, and 50 mg of CBG isolate in 30 ml of MCT oil. Weigh 400 mg of CBD isolate, 50 mg of CBC isolate, and 50 mg of CBG isolate to achieve an 8:1:1 ratio of CBD to CBC to CBG. Add the cannabinoid isolates to 30 ml of MCT oil in a mixing container. Use high-pressure homogenization or ultrasonication to mechanically process the mixture, breaking down the cannabinoids into nano-sized particles. This processing ensures that the cannabinoids are uniformly dispersed and stably suspended in the MCT oil, achieving nano-particle size for enhanced bioavailability. This 8:1:1 formulation of CBD, CBC, and CBG in MCT oil provides a well-dispersed and stable nano-suspension, optimized for rapid absorption and consistent dosing. The mechanical processing results in a homogenous formulation suitable for tinctures, capsules, or other delivery methods where precise cannabinoid ratios and stability are important.

CBG:CBD:CBDV:CBC (2:1:0.5:1:0.5) formulation in MCT oil: 200 mg of CBG isolate, 100 mg of CBD isolate, 50 mg of CBDV isolate, 100 mg of CBC isolate, and 50 mg of CBN isolate in 30 ml of MCT oil. Measure 200 mg of CBG isolate, 100 mg of CBD isolate, 50 mg of CBDV isolate, 100 mg of CBC isolate, and 50 mg of CBN isolate to achieve the 2:1:0.5:1:0.5 ratio. Add the measured cannabinoids to 30 ml of MCT oil in a mixing container. Using high-pressure homogenization or ultrasonication, mechanically process the mixture to reduce the cannabinoid particles to nano size. This processing step ensures a uniform, stable dispersion within the MCT oil, enhancing the bioavailability of the cannabinoids. This formulation provides a 2:1:0.5:1:0.5 blend of CBG, CBD, CBDV, CBC, and CBN in MCT oil, creating a nano-suspension promotes consistent dosing. The result is a stable, well-dispersed cannabinoid blend suitable for tinctures, capsules, or other applications requiring precise ratios and high bioavailability.

CBD:CBC:CBDV (5:1:1) in MCT oil: 250 mg of CBD isolate, 50 mg of CBC isolate, 50 mg of CBDV isolate, 30 ml of MCT oil. Measure out 250 mg of CBD isolate, 50 mg of CBC isolate, and 50 mg of CBDV isolate to achieve the 5:1:1 ratio. Add the cannabinoid isolates to 30 ml of MCT oil in a mixing container. Use ultrasonication or high-pressure homogenization to mechanically process the mixture, reducing the cannabinoid particles to nano size. This processing ensures a stable, nano-particle dispersion in the MCT oil, enhancing bioavailability. This 5:1:1 CBD:CBC:CBDV formulation in MCT oil provides a well-dispersed, nano-sized cannabinoid suspension, ideal for tinctures, capsules, or other delivery forms where precise ratios, stability, and enhanced absorption are desired.

The oil medium of the above examples may be replaced with any other oil that is suitable for the suspension of cannabinoids. The volume of the oil-based medium and the amount of cannabinoids can also be adjusted without affecting the effectiveness of the formulation.

While the exact anxiolytic mechanism of action of cannabidiol (CBD) is not completely understood, research has identified multiple pharmacological mechanisms through which CBD exerts its therapeutic effects. As described by Lee et al., the potential therapeutic effects of CBD have been related to multiple pharmacological mechanisms, including the agonism of 5-HT1A receptors, inhibition of reuptake and/or metabolism of the endocannabinoid anandamide (resulting indirectly in cannabinoid receptor activation), activation of transient receptor potential vanilloid 1 (TRPV1) channels, inhibition of adenosine reuptake, antagonism of GPR55, agonism of PPARY receptors, intracellular Ca2+ increase, and anti-oxidative effects, among others. However, only two of these mechanisms, 5-HTIA receptor activation and indirect potentiation of endocannabinoid transmission, have been implicated in the attenuation of defensive responses to threatening or stressful stimuli (Lee, J. L. C., Bertoglio, L. J., Guimarães, F. S., and Stevenson, C. W. (2017). Cannabidiol regulation of emotion and emotional memory processing: relevance for treating anxiety-related and substance abuse disorders. British Journal of Pharmacology 174:3242-3256).

The mechanism for the effect of CBD on serotonin 5-HTIA receptors is well-established. The 5-HT1A receptor (5-HTIAR) is an established anxiolytic target. In preclinical studies, 5-HT1AR agonists are anxiolytic in animal models of general anxiety, prevent the adverse effects of stress, and enhance fear extinction. Both pre- and postsynaptic 5-HT1ARs are coupled to various members of the Gi/o protein family. They are expressed on serotonergic neurons in the raphe, where they exert autoinhibitory function, and in various other brain areas involved in fear and anxiety. Mechanisms underlying the anxiolytic effects of 5-HTIAR activation are complex, varying between both brain region and pre-versus postsynaptic locus, and are not fully established. While in vitro studies suggest CBD acts as a direct 5-HTIAR agonist, in vivo studies are more consistent with CBD acting as an allosteric modulator, or facilitator of 5-HTIA signaling (Blessing, E. M., Steenkamp, M. M., Manzanares, J., and Marmar, C. R. (2015). Cannabidiol as a Potential Treatment for Anxiety Disorders. Neurotherapeutics 12:825-836).

The second anxiolytic mechanism is the ability of CBD to indirectly activate the cannabinoid type 1 receptor (CB1R) by inhibiting the metabolism of the endocannabinoid (eCB) anandamide. CBD inhibits both fatty acid amide hydrolase (FAAH), the enzyme which metabolizes anandamide, and fatty acid binding proteins (FABPs), which mediate the transport of anandamide to FAAH. Both mechanisms ultimately result in the indirect activation of CB1 and/or CB2 receptors. The CBIR is an inhibitory Gi/o protein-coupled receptor that is mainly localized to nerve terminals and is expressed on both γ-aminobutyric acid-ergic and glutamatergic neurons. The primary mechanism by which eCBs regulate synaptic function is retrograde signaling, wherein eCBs produced by depolarization of the postsynaptic neuron activate presynaptic CB1Rs, leading to inhibition of neurotransmitter release (Blessing et al., 2015).

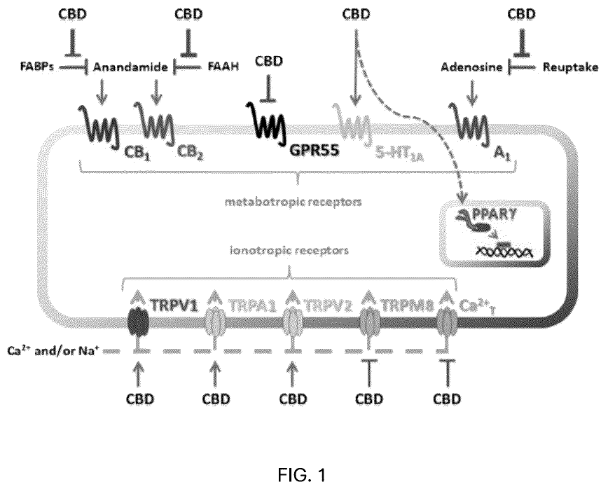

FIG. 1 provides a summary of the main molecular targets and potential mechanisms of action of CBD. As illustrated, CBD inhibits both FAAH and FABPs, both mechanisms ultimately resulting in the indirect activation of CB1 and/or CB2 receptors. CBD also activates the 5-HT1A receptor, PPARγ and the transient receptor potential channels TRPV1, TRPA1 and TRPV2. Additionally, CBD inhibits adenosine reuptake and antagonizes GPR55, TRPM8 and T-type Ca2+ channels. The 5-HT1A and (indirect) cannabinoid receptor activation are the mechanisms that have been implicated in the anxiolytic effects of CBD (Lee et al., 2017).

Many dogs suffer from anxiety-related issues, such as separation anxiety, noise phobias, and generalized anxiety. It is reported that at least two-thirds of dog-owning clients report noise averse responses to sudden sounds (Diess, F. (2023). Understanding and treating noise aversion in dogs. Zoetis Petcare). Generally, noise-related anxiety will be triggered by sounds that the dog doesn't hear frequently, such as thunder, fireworks and sirens. For some pets, even everyday household noises—vacuum cleaners are often a culprit—can be profoundly disturbing. While reactions to noise vary—trembling, hiding, lip licking, whimpering and incessant barking are common—all will alert compassionate owners to their pets' misery and the need for an effective remedy.

Currently there are two approved animal drugs for treating noise aversion in dogs. Sileo® (NADA 141-456; Zoetis) is an oromucosal gel containing dexmedetomidine and is indicated for the treatment of noise aversion in dogs. Dexmedetomidine is a potent and selective alpha-2 adrenoceptor agonist belonging to the imidazole class that inhibits the release of noradrenaline from noradrenergic neurons (Sileo (dexmedetomidine oromucosal gel) Prescribing Information. Zoetis Inc., Kalamazoo, MI. Revised 2023). This inhibition of noradrenaline release subsequently inhibits neuronal firing, which, in addition to anxiolysis, results in hypotension, bradycardia, sedation, and analgesia, which can lead to profound and undesirable side effects (Gertler, R., Brown, H. C., Mitchell, D. H., and Silvius, E. N. (2001). Dexmedetomidine: a novel sedative-analgesic agent. Proceedings (Baylor University Medical Center) 14 (1): 13-21).

The second approved animal drug for treating noise aversion in dogs is Pexion®, an oral tablet containing imepitoin (NADA 141-509; Boehringer Ingelheim), which is also an imidazole. It acts at the benzodiazepine binding site of the γ-aminobutyric acid type A (GABAA) receptor as a low-affinity partial agonist (Engel, O., Löscher, W., and Förster, C. (2019). Effects of imepitoin on seizure threshold and hippocampal neurogenesis in a mouse model of epilepsy. Epilepsia Open 4:368-380). It therefore has similar anxiolytic properties of the benzodiazepine class, which include diazepam and midazolam. Although imepitoin has been shown to be efficacious for controlling fear and anxiety associated with noise phobia in dogs, it must be administered for 2 days prior to the noise event, which may be suitable for predictable events such as New Year's Eve and Fourth of July but is very impractical for many other noise-related situations such as thunderstorms.

The cannabinoid-based formulations described in the present invention offer a novel mechanism of action compared to currently approved treatments, avoiding the sedative and cardiovascular side effects associated with alpha-2 adrenoceptor agonists and the requirement for advance dosing associated with GABAA receptor partial agonists.

CHAN-001: Novel CBD Formulation for Noise Aversion

In a specific embodiment, the present invention provides CHAN-001, a novel cannabidiol (CBD) oral suspension specifically developed for the treatment of noise aversion in dogs. CHAN-001 represents a significant advancement in veterinary cannabinoid therapeutics through its unique combination of ultra-high purity CBD isolate and nano-particle formulation.

CHAN-001 utilizes a proprietary process to obtain highly pure (>99.99%) hemp-derived CBD isolate as the active pharmaceutical ingredient (API). This extreme purity level, exceeding even the >99.9% purity standards described elsewhere in this specification, ensures exceptional consistency and reliability in therapeutic response. The manufacturing of API with tested and validated consistency is critical for the demonstration of an appropriate dose response and drug approval. In comparison, typical “bulk” isolates used as fillers in other formulations do not achieve this level of pharmaceutical-grade purity.

The CBD isolate in CHAN-001 is processed through nanotization to reduce particle size to nanoscale dimensions, typically less than 100 nanometers, preferably less than 80 nanometers, and most preferably between 20 and 60 nanometers. This nanotization of particle size increases the ability of the API to penetrate the blood-brain barrier, which is particularly advantageous for treating neurological and behavioral conditions such as noise aversion and anxiety. The combination of extremely high purity levels of API in addition to nanotized particle size results in lower dose levels needed to achieve the therapeutic effect compared to conventional CBD formulations.

The mechanism of action of CHAN-001 is based on the anxiolytic properties of CBD, which operates through two primary pathways as illustrated in FIG. 1. First, CBD acts as an agonist or allosteric modulator of the 5-HT1A serotonin receptor, which is an established anxiolytic target. Second, CBD indirectly activates cannabinoid receptors (CB1 and CB2) by inhibiting the metabolism of the endocannabinoid anandamide through inhibition of FAAH and FABPs. These mechanisms work synergistically to attenuate defensive responses to threatening or stressful stimuli, such as loud noises, without the sedative effects associated with other approved treatments for noise aversion (Lee et al., 2017; Blessing et al., 2015).

CHAN-001 is formulated as an oral suspension in an oil-based medium, preferably medium-chain triglyceride (MCT) oil, at a concentration of approximately 1 mg/ml. The dosage for CHAN-001 typically ranges from 0.5 mg to 10 mg per kg of body weight, with preferred dosing at approximately 1 mg/kg for acute noise aversion events. The formulation is designed for rapid absorption through the oral mucosa and esophagus, with administration preferably 20 minutes prior to an anticipated noise event, though it may be administered from 60 minutes before to immediately after the onset of the noise event.

The safety profile of CHAN-001 is expected to be superior to currently approved treatments for noise aversion. Studies have demonstrated that daily long-term administration of a broad-spectrum CBD product dosed at 4 mg/kg/day over 26 weeks was well tolerated in dogs (Bradley, S., Shreyer, T., Cital, S., Kogan, L., and Heather, K. (2022). Safety evaluation of a hemp-derived cannabidiol product in healthy dogs. Frontiers in Veterinary Science 9:988903). Additionally, exposure to CBD or a combination of CBD plus other cannabinoids at 5 mg/kg over 90 days was well tolerated (Bookout, W., Highet, B., Goerl, K., and Miller, A. (2024). A 90-day toxicity study of cannabidiol and cannabidiol/49-tetrahydrocannabinol (20:1) oral preparations in healthy Beagle dogs. Frontiers in Veterinary Science 11:1347456). The lower dose requirements achieved through the nano-particle formulation and ultra-high purity of CHAN-001 further enhance the safety profile by reducing the potential for side effects. A known side effect of high CBD doses in mammals is an elevated liver enzyme response; the reduction in required dose yields a corresponding reduction in this side effect. Furthermore, CHAN-001 avoids the hypotension, bradycardia, and profound sedation associated with alpha-2 adrenoceptor agonists like dexmedetomidine, and does not require the advance dosing protocol required by GABAA receptor partial agonists like imepitoin.

The unique advantages of CHAN-001 include: (1) a novel mechanism of action compared to other currently approved animal drugs for noise aversion, operating through 5-HT1A receptor modulation and indirect cannabinoid receptor activation rather than alpha-2 adrenoceptor agonism or GABAA receptor modulation; (2) a unique formulation with ultra-high API purity (>99.99%); (3) novel nanotization of particle size which improves blood-brain barrier penetration and therapeutic effects at lower doses; (4) an improved safety profile with fewer side effects, specifically avoiding lethargy and sedation; and (5) flexible dosing that allows administration immediately prior to or during a noise event, rather than requiring days of advance dosing.

### Exemplary Clinical Protocol for the Treatment of Noise Aversion in Non-Human Mammals

This clinical study aims to evaluate the safety and efficacy of CHAN-001, a veterinarian medicine composition, for treating noise aversion in non-human mammals, specifically canines. The study will focus on administering a CBD isolate nano-particle formulation in an oil-based medium, which contains CBD with a purity level exceeding 99% and a particle size of less than 100 nanometers. The formulation is essentially free of THC, making it non-psychoactive and suitable for use in animals. The study is designed as a randomized, double-blind, placebo-controlled pilot trial conducted in a field and laboratory setting.

A total of 68 healthy canines, aged between 2 and 5 years and weighing between 5 and 40 kg, will be enrolled. These canines, each demonstrating a history of acute noise aversion behaviors (such as whining, trembling, hiding, or attempts to escape) in response to loud noises, will be divided into two cohorts. Each cohort will be further randomized into treatment and control groups, ensuring a blinded format. The treatment group in Cohort #1 will receive a low dose of CHAN-001 at a concentration at 1 mg/kg, while Cohort #2 will receive a higher dose, set at ten times the concentration of Cohort #1. The corresponding placebo groups in each cohort will receive a placebo oral solution matching the volume of the treatment doses. All solutions will be administered orally with a gradient syringe, with the preferred administration time being 20 minutes prior to the noise event.

For each cohort, noise stimuli will be provided in a controlled setting by setting off fireworks, using five 500-gram fireworks cakes as the standardized noise source. This controlled noise event is designed to simulate real-life noise aversion triggers, allowing for consistent and reliable evaluation of the canines' behavioral responses. The primary outcomes for this study will involve observing the canines' responses to the fireworks event, assessing specific noise aversion behaviors and comparing each animal's response with its baseline behavior when exposed to similar noise stimuli in the past. Using a standardized rubric, study monitors will categorize behavioral responses, including indicators of stress such as whining, trembling, hiding, or escape attempts. Observations will be rated as “worse,” “none,” “some,” or “good effect,” and effectiveness will be evaluated at least three hours after the last dose of CHAN-001 or placebo.

Adverse reactions will be closely monitored throughout the study to ensure the safety of all subjects. Specific adverse reactions to be recorded include excessive sedation, agitation, gastrointestinal issues, respiratory distress, neurological symptoms (such as tremors or seizures), skin reactions, changes in appetite, and excessive drooling. If a study subject exhibits an unusual behavior that does not align with the predefined adverse reaction categories, that subject will be removed from the study to maintain data integrity. Each observation will be documented with the date and time, a description of the behavior, the duration of the reaction, a severity rating, and any actions taken (such as discontinuing treatment or seeking veterinary assistance).

CHAN-001 is a nanoparticle suspension of CBD as describe herein. CHAN-001 is typically formulated in an oil base, such as MCT, at the concentration of about 1 mg/ml. The dosage for CHAN-001 will vary within a range of 0.5 mg to 10 mg per kg of body weight, depending on the cohort assignment. This range is expected to optimize bioavailability while minimizing the risk of adverse effects. While the preferred administration time is 20 minutes prior to the noise event, the study will also evaluate efficacy when administered at specific intervals up to 60 minutes before, as close as 5 minutes prior, or even after the noise event, to assess both preventative and responsive treatment outcomes.

This protocol ensures a structured approach to assessing both the safety and therapeutic potential of CHAN-001 in managing acute noise aversion in canines. By using a controlled noise event with fireworks and specifying a preferred administration time, this study offers insights into the formulation's efficacy in reducing noise-induced anxiety and stress behaviors, providing an evidence-based approach to treating noise aversion in animals.

### CHAN-002: Novel CBD Formulation for Separation Anxiety

Separation anxiety (SA) is a prevalent and debilitating condition affecting companion animals, particularly dogs. It is estimated to impact around 14 percent of dogs, with affected animals displaying profound distress when left alone or separated from their primary caregiver. Unlike simple behavioral issues, separation anxiety is characterized by an inability to self-soothe or find comfort when alone, leading to destructive behaviors such as urinating or defecating indoors, chewing furniture, or vocalizing excessively. This is a serious condition that significantly impacts both animal welfare and the human-animal bond.

### Currently Approved Treatments for Separation Anxiety in Dogs

Current veterinary products approved for separation anxiety in dogs include Reconcile® (fluoxetine, NADA 141-272) and Clomicalm® (clomipramine, NADA 141-120). Fluoxetine is a selective serotonin reuptake inhibitor (SSRI) that is widely used in human medicine as an antidepressant and subsequently approved for use in dogs for separation anxiety. It acts by binding to the serotonin transporter protein and therefore blocking serotonin (5-hydroxytryptamine; 5-HT) reuptake from the pre-synaptic clefts in the central nervous system. This results in greater levels of serotonin in the brain, which can improve message signaling. However, side effects of fluoxetine can include seizures, lethargy, vomiting, diarrhea, and tremors, as well as transient hyporexia and weight loss.

Clomipramine is a tricyclic antidepressant, or a serotonin-norepinephrine reuptake inhibitor (SNRI) that inhibits serotonin reuptake in the synaptic clefts, in addition to blocking the reuptake of norepinephrine. This results in increased serotonergic and noradrenergic neurotransmission. However, side effects such as sedation, lethargy, depression, hypotension, anorexia, and dry mouth can occur due to the antagonism of the H1, al-adrenergic, and muscarinic acetylcholine receptors. Additional adverse effects include anorexia, vomiting, elevation in liver enzymes, and impaired synthesis of thyroxine.

The cannabinoid-based formulations described in the present invention offer a novel mechanism of action compared to currently approved treatments for separation anxiety, avoiding the systemic side effects associated with SSRIs and SNRIs while providing anxiolytic effects through 5-HT1A receptor modulation and indirect cannabinoid receptor activation.

In a specific embodiment, the present invention provides CHAN-002, a novel cannabidiol (CBD) oral suspension specifically developed for the treatment of separation anxiety in dogs. CHAN-002 represents a significant advancement in veterinary cannabinoid therapeutics through its unique combination of ultra-high purity CBD isolate and nano-particle formulation designed for daily administration to provide consistent management of chronic separation anxiety symptoms.

CHAN-002 utilizes a proprietary process to obtain highly pure (>99.99%) hemp-derived CBD isolate as the active pharmaceutical ingredient (API). This extreme purity level, exceeding even the >99.9% purity standards described elsewhere in this specification, ensures exceptional consistency and reliability in therapeutic response. The manufacturing of API with tested and validated consistency is critical for the demonstration of an appropriate dose response and drug approval. In comparison, typical “bulk” isolates used as fillers in other formulations do not achieve this level of pharmaceutical-grade purity.

The CBD isolate in CHAN-002 is processed through nanotization to reduce particle size to nanoscale dimensions, typically less than 100 nanometers, preferably less than 80 nanometers, and most preferably between 20 and 60 nanometers. This nanotization of particle size increases the ability of the API to penetrate the blood-brain barrier, which is particularly advantageous for treating chronic neurological and behavioral conditions such as separation anxiety. The combination of extremely high purity levels of API in addition to nanotized particle size results in lower dose levels needed to achieve the therapeutic effect compared to conventional CBD formulations.

The mechanism of action of CHAN-002 is based on the anxiolytic properties of CBD, which operates through two primary pathways as illustrated in FIG. 1. First, CBD acts as an agonist or allosteric modulator of the 5-HTIA serotonin receptor, which is an established anxiolytic target. Second, CBD indirectly activates cannabinoid receptors (CB1 and CB2) by inhibiting the metabolism of the endocannabinoid anandamide through inhibition of FAAH and FABPs (Lee, J. L. C., Bertoglio, L. J., Guimarães, F. S., and Stevenson, C. W. (2017). Cannabidiol regulation of emotion and emotional memory processing: relevance for treating anxiety-related and substance abuse disorders. British Journal of Pharmacology 174:3242-3256; Blessing, E. M., Steenkamp, M. M., Manzanares, J., and Marmar, C. R. (2015). Cannabidiol as a Potential Treatment for Anxiety Disorders. Neurotherapeutics 12:825-836).

Unlike SSRIs such as fluoxetine, which block serotonin reuptake at the transporter protein, or SNRIs such as clomipramine, which block both serotonin and norepinephrine reuptake, CBD's mechanism of action involves modulation of the 5-HT1A receptor itself rather than manipulation of neurotransmitter reuptake. This distinction in mechanism of action is illustrated in FIG. 1 and results in a different side effect profile. While SSRIs and SNRIs can cause systemic effects including seizures, lethargy, gastrointestinal disturbances, and impacts on liver function and thyroid hormone synthesis, CBD's targeted receptor modulation and indirect cannabinoid activation provide anxiolytic effects without these systemic complications.

CHAN-002 is formulated as an oral suspension in an oil-based medium, preferably medium-chain triglyceride (MCT) oil, at a concentration of approximately 1 mg/ml. The dosage for CHAN-002 typically ranges from 0.25 mg to 10 mg per kg of body weight, with preferred dosing for chronic separation anxiety management starting at lower doses (0.25-1 mg/kg) and escalating as needed based on individual patient response. CHAN-002 is administered daily to provide consistent therapeutic levels for ongoing management of separation anxiety behaviors. The formulation is designed for rapid absorption through the oral mucosa and esophagus, with administration preferably 20 minutes prior to owner departure to achieve peak therapeutic levels during the separation period.

The safety profile of CHAN-002 is expected to be superior to currently approved treatments for separation anxiety. Studies have demonstrated that daily long-term administration of a broad-spectrum CBD product dosed at 4 mg/kg/day over 26 weeks was well tolerated in dogs (Bradley, S., Shreyer, T., Cital, S., Kogan, L., and Heather, K. (2022). Safety evaluation of a hemp-derived cannabidiol product in healthy dogs. Frontiers in Veterinary Science 9:988903). Additionally, exposure to CBD or a combination of CBD plus other cannabinoids at 5 mg/kg over 90 days was well tolerated (Bookout, W., Highet, B., Goerl, K., and Miller, A. (2024). A 90-day toxicity study of cannabidiol and cannabidiol/49-tetrahydrocannabinol (20:1) oral preparations in healthy Beagle dogs. Frontiers in Veterinary Science 11:1347456). These long-term safety studies are particularly relevant for CHAN-002 given its intended use for chronic daily administration in the management of separation anxiety.

The lower dose requirements achieved through the nano-particle formulation and ultra-high purity of CHAN-002 further enhance the safety profile by reducing the potential for side effects. A known side effect of high CBD doses in mammals is an elevated liver enzyme response; the reduction in required dose yields a corresponding reduction in this side effect. Furthermore, CHAN-002 avoids the seizures, lethargy, vomiting, diarrhea, tremors, hyporexia, and weight loss associated with SSRIs like fluoxetine, as well as the sedation, lethargy, depression, hypotension, anorexia, dry mouth, elevation in liver enzymes, and impaired thyroid hormone synthesis associated with SNRIs like clomipramine.

The unique advantages of CHAN-002 include: (1) a novel mechanism of action compared to other currently approved animal drugs for separation anxiety, operating through 5-HTIA receptor modulation and indirect cannabinoid receptor activation rather than serotonin or serotonin-norepinephrine reuptake inhibition; (2) a unique formulation with ultra-high API purity (>99.99%); (3) novel nanotization of particle size which improves blood-brain barrier penetration and therapeutic effects at lower doses; (4) an improved safety profile with fewer side effects compared to SSRIs and SNRIs, specifically avoiding gastrointestinal disturbances, seizure risk, and metabolic complications; (5) suitability for long-term daily administration in the chronic management of separation anxiety; and (6) rapid onset of action allowing for administration shortly before owner departure rather than requiring weeks of loading as with traditional antidepressant medications.

Clinical protocols for evaluating CHAN-002 in dogs with separation anxiety involve dose-escalation studies to determine optimal tolerability and efficacy, followed by evaluation of steady-state pharmacokinetics and long-term safety and efficacy compared to existing treatments such as fluoxetine. The dose-escalation approach allows for individualized dosing based on patient response and minimizes the risk of adverse effects during initial treatment phases.

### Exemplary Clinical Protocol for the Treatment of Separation Anxiety in Non-Human Mammals

The primary objective of this study is to conduct a dose-escalation trial to determine the tolerability of CHAN-002 in healthy dogs. This study will investigate different dose levels to identify a dose that is well-tolerated and biologically effective. Once this optimal dose is determined, it will be used to enroll a new cohort of dogs to evaluate the steady-state pharmacokinetics (PK) of CHAN-002. The data collected from this study will inform future clinical studies on the chronic tolerability and efficacy of CHAN-002 in dogs with separation anxiety (SA). Additionally, this data may support further studies on the clinical effects of CHAN-002 in dogs with SA, potentially comparing its efficacy to existing treatments like Fluoxetine.

### Study Design

This multi-center, double-masked, controlled study aims to assess the safety and efficacy of CHAN-002 in managing separation anxiety behaviors. Initially, three pilot non-clinical laboratory studies will be conducted with 34 healthy canines to observe any excessive sedation or abnormal side effects. CHAN-002 will be administered orally at a dosage of 1 mg per kg of body weight, 20 minutes before the owner's departure.

In the main study, 68 healthy dogs, aged 2 to 10 years and weighing between 5 and 80 kg, will be enrolled. These dogs will undergo a 14-day pre-treatment observation period, during which a board-certified behaviorist will document SA-related behaviors, including destructive actions, excessive vocalization, inappropriate elimination, excessive salivation, grooming, shaking, restlessness, and signs of depression. Following this period, each dog will be randomized into one of two cohorts and either a treatment or placebo group in a blinded format.

CHAN-001 is a nanoparticle suspension of CBD as describe herein. CHAN-002 is typically formulated in an oil base, such as MCT, at the concentration of about 1 mg/ml. The study will evaluate CHAN-002 at doses ranging from 0.25 mg to 10 mg per kg of body weight, administered 20 minutes prior to the owner's departure for a 4-hour separation period. Cohort #1 will receive an initial dose of 0.25 mg of CHAN-002, while Cohort #2 will receive a higher dose of 0.5 mg. After a 24-hour washout period, Cohort #1 will escalate to a 1 mg dose, while Cohort #2 will escalate to a 5 mg dose. A final dose escalation will occur after an additional 24 hours, with Cohort #1 receiving 10 mg of CHAN-002.

Each separation event will include either CHAN-002 or a placebo in the same oil-based solution without active ingredients, administered 20 minutes prior to owner departure. Dogs will be exposed to separation conditions designed to mimic real-life triggers of separation anxiety. Initial treatments will be placebo-based to establish a behavioral baseline, followed by incremental CHAN-002 dosing. Behavioral responses will be assessed according to a standardized rubric that evaluates SA behaviors relative to historical baselines.

### Safety and Tolerability Monitoring

Safety and tolerability will be closely monitored throughout the study by the behaviorist and veterinary team, noting adverse reactions such as excessive sedation, agitation, gastrointestinal issues, respiratory distress, neurological symptoms, skin reactions, changes in appetite, and excessive drooling. Any dog exhibiting behaviors not aligned with the predefined adverse reaction categories will be removed from the study to maintain data integrity. Observations will document the time, description, duration, and severity of each reaction, as well as any interventions taken, such as discontinuing treatment or seeking veterinary assistance.

### Owner Involvement and Observation

Dog owners will administer the study medication daily, implement a behavior modification plan, record observations, and complete weekly questionnaires. Dogs that were crated prior to the study to manage SA will continue to be crated to ensure consistency. The investigator will perform physical examinations mid-study (Day 42) and at the study's conclusion (Day 70). Owners will return daily logs and any remaining medication at the end of the treatment period.

### Exemplary Clinical Protocol for the Treatment of Seizures and Idiopathic Epilepsy in Non-Human Mammals

Title: Evaluation of Cannabinoid Treatment for Seizures and Idiopathic Epilepsy in Cats, Dogs, and Horses.

Study Objective: To evaluate the efficacy and safety of a cannabinoid composition, including cannabidiol (CBD), and/or cannabidivarin (CBDV), and other cannabinoids, in reducing the frequency and severity of seizures in non-human mammals diagnosed with idiopathic epilepsy.

Study Design: A multicenter, randomized, double-blind, placebo-controlled clinical trial.

Duration: 12 weeks

Participants: Non-human mammals (cats, dogs, and horses) diagnosed with idiopathic epilepsy.

Inclusion Criteria: 1. Diagnosis: Confirmed diagnosis of idiopathic epilepsy by a veterinary neurologist, with a history of recurrent seizures (at least 2 seizures per month) for a minimum of 3 months. 2. Age: Participants aged between 6 months to 8 years. 3. Weight: Minimum weight of 5 kg. 4. Health Status: General health assessed via a thorough physical examination, with no significant concurrent diseases. 5. Owner Consent: Written informed consent obtained from the owner for participation in the study.

Exclusion Criteria: 1. Secondary Epilepsy: Presence of seizures due to structural brain lesions or other identifiable causes (e.g., metabolic disorders). 2. Concurrent Medications: Use of other antiepileptic drugs or medications that may interfere with study outcomes within the last month. 3. Hypersensitivity: Known hypersensitivity or adverse reactions to cannabinoids. 4. Severe Health Issues: Significant hepatic or renal impairment, severe cardiovascular disease, or any condition that may complicate participation. 5. Pregnant or Lactating: Pregnant or lactating females are excluded.

Treatment Groups: 1. Treatment Group: Cannabinoid composition administered in a form selected from the group consisting of: Orally dosed liquid, orally dosed oil, orally dosed gel, orally dosed tablet, orally dosed chew. 2. Control Group: Placebo administered in the same orally dosed form.

Dosage and Treatment Regimen: Initial Dosage: Administer an initial dose of 2 mg/kg of body weight per day. Administration Schedule: All participants to receive their assigned treatment once daily at the same time each day. The owners are instructed to maintain a dosing log to track administration.

Clinical Evaluation: 1. Baseline Assessments: Comprehensive neurological examination, Detailed medical history, and Seizure frequency and duration log maintained for 2 weeks prior to the study; Behavioral assessments (baseline anxiety, activity level).

2. Follow-Up Assessments (at 4, 8, and 12 weeks): Neurological examination by a veterinarian, Seizure frequency and duration log maintained by the owner, Clinical evaluation of seizure severity using a standardized scale: Mild: Short duration, infrequent; Moderate: Longer duration, more frequent, some postictal signs; Severe: Prolonged duration, clusters of seizures, significant postictal signs. Owner questionnaires assessing behavioral changes, side effects, and overall well-being.

Primary Outcome Measures: Seizure Frequency: Change in the number of seizures per month from baseline to the end of the study (12 weeks). Seizure Severity: Change in seizure severity scores as assessed at each follow-up.

Secondary Outcome Measures: Changes in overall behavioral assessments (anxiety, activity level). Incidence of any adverse events and side effects, categorized as mild, moderate, or severe.

Safety Monitoring: Regular veterinary check-ups for physical examinations and laboratory tests (liver and kidney function) at baseline, 6 weeks, and end of the study. Immediate reporting and evaluation of any adverse reactions or side effects.

Data Analysis: Statistical analysis will be performed using appropriate methods (e.g., ANOVA for repeated measures, t-tests) to compare changes in seizure frequency and severity between treatment and control groups.

Ethical Considerations: Approval from the institutional animal care and use committee (IACUC). Informed consent obtained from all participants' owners. Adherence to the principles of humane treatment and welfare of non-human mammals.

Idiopathic epilepsy (IE) represents a chronic neurological condition characterized by seizures without recognizable intracranial or extracranial abnormalities. This condition is the most prevalent neurological ailment in dogs, impacting an estimated 0.5% to 5.7% of the overall canine population. The more severe expressions of the disease frequently lead to euthanasia due to concerns about the quality of life, underscoring the importance of effective seizure control and management.

Various anti-seizure medications (ASM) are employed for IE management, with the current consensus statement from the American College of Veterinary Internal Medicine advocating phenobarbital and potassium bromide as primary options for initial management. However, these medications may prove ineffective in controlling seizures for up to 30% of epileptic dogs. Moreover, these conventional treatments often come with associated adverse events (AE), emphasizing the necessity for alternative therapeutic approaches. Neither of these products are fully approved by the Center for Veterinary Medicine (CVM) for use in dogs.

Phenobarbital is currently the first-line treatment of choice for treating canine epilepsy. However, phenobarbital is known to interact with many other veterinary drugs, including but not limited to lysodren, corticosteroids, beta blockers, cyclosporine, levetiracetam, praziquantel, itraconazole, and metronidazole (Brooks W. Phenobarbital. Veterinary Partner®. (2024). Website: https://veterinarypartner.vin.com/default.aspx?pid=19239&id=4951404). Phenobarbital side effects include sedation, excessive appetite and thirst, and following chronic exposure can lead to irreversible scarring of the liver and liver failure (Brooks, 2024). These effects result in the need for veterinarians to frequently monitor clinical pathology parameters (particularly those related to liver function) in addition to therapeutic monitoring of phenobarbital blood levels.

Recent clinical studies have demonstrated the potential of cannabinoids for treating refractory epileptic seizures in dogs. McGrath et al. conducted a randomized blinded controlled clinical trial to assess the effect of oral cannabidiol administration in addition to conventional antiepileptic treatment on seizure frequency in dogs with intractable idiopathic epilepsy, finding a significant reduction in seizure frequency (McGrath S, Bartner L R, Rao S, Packer R A, Gustafson D L. Randomized blinded controlled clinical trial to assess the effect of oral cannabidiol administration in addition to conventional antiepileptic treatment on seizure frequency in dogs with intractable idiopathic epilepsy. JAVMA (2019) 254:1301-1308). Subsequently, Rozental et al. demonstrated the efficacy and safety of cannabidiol as adjunct treatment for drug-resistant idiopathic epilepsy in 51 dogs through a double-blinded crossover study (Rozental A J, Weisbeck B G, Alvarenga I C, Gustafson D L, Kusick B R, Rao S, Bartner L R, McGrath S. The efficacy and safety of cannabidiol as adjunct treatment for drug-resistant idiopathic epilepsy in 51 dogs: A double-blinded crossover study. J Vet Intern Med. (2023) 37:2291-2300). Garcia et al. further evaluated the safety and efficacy of cannabidiol-cannabidiolic acid rich hemp extract in the treatment of refractory epileptic seizures in dogs (Garcia G A, Kube S, Carrera-Justiz S, Tittle D, Wakshlag J J. Safety and efficacy of cannabidiol-cannabidiolic acid rich hemp extract in the treatment of refractory epileptic seizures in dogs. Front Vet Sci. (2022) 9:939966).

The cannabinoid-based formulations described in the present invention offer a novel mechanism of action compared to currently available treatments for idiopathic epilepsy, avoiding the hepatotoxicity, drug interactions, and need for frequent therapeutic monitoring associated with phenobarbital.

In a specific embodiment, the present invention provides CHIE-001, a novel cannabinoid oral suspension specifically developed for controlling seizures associated with idiopathic epilepsy in dogs. CHIE-001 represents a significant advancement in veterinary anti-seizure therapeutics as the first formulation to combine three highly purified phytocannabinoid isolates—cannabidiol (CBD), cannabidivarin (CBDV), and cannabichromene (CBC)—in a precisely controlled ratio for therapeutic use.

CHIE-001 comprises CBD isolate, CBDV isolate, and CBC isolate in a weight ratio of 5:1:1, suspended in an oil-based medium. This specific ratio has been optimized to provide synergistic anti-seizure effects through complementary mechanisms of action. Each cannabinoid isolate is obtained through a proprietary process to achieve ultra-high purity (>99.99%) from hemp-derived sources. This extreme purity level, exceeding even the >99.9% purity standards described elsewhere in this specification, ensures exceptional consistency and reliability in therapeutic response. The manufacturing of API with tested and validated consistency is critical for the demonstration of an appropriate dose response and drug approval.

Table 1 shows an exemplary CHIE-001 cannabinoids analysis. In the particular sample tested total THC is at 0.01%. Note: ND=Not Detected; <LOQ=Below Limit of Quantification; LOD>0.005% by mass; LOQ>0.01% by mass; Total THC is calculated by Δ9-THC+ (THCA-A×0.877) and Total CBD is calculated by CBD+ (CBDA×0.877).

TABLE 1
Exemplary CHIE-001 cannabinoids analysis

| Cannabinoid | RT | Total % |
|---|---|---|
| Cannabidivarinic Acid (CBDVA) | ND | ND |
| Cannabidivarin (CBDV) | 2.33 | 1.06 |
| Cannabidiolic Acid (CBDA) | ND | ND |
| Cannabigerolic Acid (CBGA) | ND | ND |
| Cannabinol (CBN) | ND | ND |
| Cannabidiol (CBD) | 3.46 | 5.06 |
| Cannabigerol (CBG) | <LOQ | <LOQ |
| Tetrahydrocannabivarin (THCV) | ND | ND |
| Tetrahydrocannabivarin Acid (THCVA) | ND | ND |
| Delta-9-Tetrahydrocannabinol (Δ9-THC) | 6.56 | 0.01 |
| Delta-8-Tetrahydrocannabinol (Δ8-THC) | 6.74 | 0.04 |
| Tetrahydrocannabinolic acid (THCA-A) | ND | ND |
| Cannabichromene (CBC) | 8.18 | 0.98 |
| Cannabichromene Acid (CBCA) | ND | ND |
| Δ10 and Δ6a,10a-Tetrahydrocannabinol | ND | ND |
| Cannabicitran (CBTC) | ND | ND |

Each of the three cannabinoid isolates in CHIE-001 is processed through nanotization to reduce particle size to nanoscale dimensions, typically less than 100 nanometers, preferably less than 80 nanometers, and most preferably between 20 and 60 nanometers. This nanotization of particle size increases the ability of the APIs to penetrate the blood-brain barrier, which is essential for treating neurological conditions such as epilepsy. The combination of extremely high purity levels of APIs in addition to nanotized particle size results in lower dose levels needed to achieve the therapeutic effect compared to conventional cannabinoid formulations.

While the exact mechanism of cannabinoids for reducing seizure activity in human and veterinary patients is not fully known, data indicate that these cannabinoids are mimicking and modulating the γ-aminobutyric acid (GABA) pathway. GABA is the primary inhibitory neurotransmitter in the brain, which acts to counterbalance the excitation of neurons. The effect of GABA and GABA receptors has been well studied. When GABA is released into the synapse, it interacts with GABAA receptors to control chloride entry into the cell and GABAB receptors to decrease calcium entry and inhibit pre-synaptic release of other transmitters, specifically impacting the inhibitory function of mature central neurons.

The proposed anti-seizure mechanism of action for CBD includes reducing presynaptic intracellular calcium concentrations, which limits neuronal excitability and subsequently prevents excessive neurotransmitter release (Potschka H, Bhatti S F M, Tipold A, McGrath S. Cannabidiol in canine epilepsy. Vet Journal. (2022) 290:105913). This mechanism is distinct from the mechanisms of action of phenobarbital or potassium bromide, which elicit their effects through changes in chloride ion potentials.

Recent research has demonstrated that cannabichromene (CBC) exerts acute anti-seizure effects through allosteric activation of GABAA receptors. Wang et al. demonstrated that CBC from full-spectrum hemp extract can allosterically activate GABAA receptors, providing a complementary mechanism to CBD's effects on calcium channel modulation (Wang Z, Zheng H, Yang H, Song H, Lian J, Peng C, Wang H, Zhang H, Zheng Y, Wang Q, Lan L, Duan G, Ma L, Peng X, Huang Z. Cannabichromene from full-spectrum hemp extract exerts acute anti-seizure effects through allosteric activation of GABAA receptors. Fundamental Res. (2024) 4:1357-1364).

Cannabidivarin (CBDV) has also demonstrated anti-seizure properties in clinical studies. Hurley et al. evaluated the efficacy and safety of cannabidivarin treatment of epilepsy in girls with Rett syndrome in a phase 1 clinical trial, demonstrating both safety and preliminary efficacy (Hurley E N, Ellaway C J, Johnson A M, Truong L, Gordon R, Galettis P, Martin J H, Lawson J A. Efficacy and safety of cannabidivarin treatment of epilepsy in girls with Rett syndrome: A phase 1 clinical trial. Epilepsia. (2022) 63:1736-1747). Additionally, minor cannabinoids including CBDV may activate transient receptor potential (TRP) ion channels and affect the excitability of the nervous system, providing yet another complementary mechanism for seizure control.

The 5:1:1 ratio of CBD:CBDV:CBC in CHIE-001 was selected to optimize the synergistic effects of these three complementary mechanisms: (1) CBD's modulation of calcium channels and reduction of presynaptic intracellular calcium concentrations; (2) CBC's allosteric activation of GABAA receptors; and (3) CBDV's effects on TRP ion channels and neuronal excitability. This multi-mechanistic approach provides broader anti-seizure coverage compared to single-cannabinoid formulations.

CHIE-001 is formulated as an oral suspension in an oil-based medium, preferably medium-chain triglyceride (MCT) oil, with the combined cannabinoid content typically at a concentration suitable for once or twice daily administration. The dosage for CHIE-001 typically ranges from 2 mg to 10 mg per kg of body weight per day of total cannabinoids, administered once daily at the same time each day to maintain consistent therapeutic levels. The formulation is designed for enhanced absorption through the oral mucosa and esophagus, ensuring rapid uptake and consistent bioavailability.

The safety profile of CHIE-001 is expected to be superior to currently available treatments for idiopathic epilepsy in dogs. Studies have demonstrated that daily long-term administration of a broad-spectrum CBD product dosed at 4 mg/kg/day over 26 weeks was well tolerated in dogs (Bradley S, Young S, Bakke A M, Holcombe L, Waller D, Hunt A, Pinfold K, Watson P, Logan D W. Long-term daily feeding of cannabidiol is well-tolerated by healthy dogs. Front Vet Sci. (2022) 9:977457). Additionally, Bookout et al. demonstrated that exposure to CBD or a combination of CBD plus other cannabinoids at 5 mg/kg over 90 days was well tolerated (Bookout W, Dziwenka M, Valm K, Kovacs-Nolan J. Safety study of cannabidiol products in healthy dogs. Front Vet Sci. (2024) 11:1349590). These long-term safety studies support the use of cannabinoid combinations for chronic daily administration in the management of idiopathic epilepsy.

The lower dose requirements achieved through the nano-particle formulation and ultra-high purity of CHIE-001 further enhance the safety profile by reducing the potential for side effects. A known side effect of high CBD doses in mammals is an elevated liver enzyme response; the reduction in required dose yields a corresponding reduction in this side effect. An inverse example on the human side is the product Epidiolex, which requires extremely high doses that subsequently flood the patient's synaptic system. The optimized formulation of CHIE-001 avoids this excessive dosing requirement.

Furthermore, CHIE-001 avoids the sedation, polyphagia, polydipsia, hepatotoxicity, and drug interactions associated with phenobarbital. Unlike phenobarbital, which requires frequent therapeutic drug monitoring and clinical pathology assessments to monitor liver function, CHIE-001 is expected to require less intensive monitoring, reducing the burden on veterinarians and pet owners while improving patient quality of life.

The unique advantages of CHIE-001 include: (1) a novel multi-cannabinoid formulation that is the first to combine CBD, CBDV, and CBC as active pharmaceutical ingredients in veterinary medicine; (2) a novel mechanism of action involving multiple complementary pathways including calcium channel modulation, GABAA receptor allosteric activation, and TRP ion channel modulation, distinct from the chloride ion potential changes induced by phenobarbital; (3) ultra-high API purity (>99.99%) for each cannabinoid component; (4) novel nanotization of particle size which improves blood-brain barrier penetration and therapeutic effects at lower doses; (5) an improved safety profile with reduced hepatotoxicity, fewer drug interactions, and less need for therapeutic and clinical pathology monitoring compared to phenobarbital; (6) efficacy in drug-resistant epilepsy cases where conventional treatments have failed; and (7) the optimized 5:1:1 ratio of CBD:CBDV:CBC providing synergistic anti-seizure effects.

Clinical protocols for evaluating CHIE-001 in dogs with idiopathic epilepsy involve multicenter, randomized, double-blind, placebo-controlled trials measuring changes in seizure frequency and severity over 12-week treatment periods, with comprehensive safety monitoring including physical examinations and laboratory tests for liver and kidney function.

This application incorporates by reference U.S. Provisional Patent Application No. 63/566,540, titled “Compositions and Method of Use of Hemp Derived Cannabinoids,” filed on Mar. 18, 2024. All documents mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### REFERENCES

1. Bialer M, Perucca E. Does cannabidiol have antiseizure activity independent of its interactions with clobazam? An appraisal of the evidence from randomized controlled trials. Epilepsia. 2020; 61:1082-1089.
2. Blessing E M, Steenkamp M M, Manzanares J, Marmar C R. Cannabidiol as a Potential Treatment for Anxiety Disorders. Neurotherapeutics. 2015; 12:825-836.
3. Bookout W, Dziwenka M, Valm K, Kovacs-Nolan J. Safety study of cannabidiol products in healthy dogs. Front Vet Sci. 2024; 11:1349590.
4. Bradley S, Young S, Bakke A M, Holcombe L, Waller D, Hunt A, Pinfold K, Watson P, Logan D W. Long-term daily feeding of cannabidiol is well-tolerated by healthy dogs. Front Vet Sci. 2022; 9:977457.
5. Brioschi F A, Di Cesare F, Gioeni D, Rabbogliatti V, Ferrari F, D′Urso E S, Amari M, Ravasio G. Oral Transmucosal Cannabidiol Oil Formulation as Part of a Multimodal Analgesic Regimen: Effects on Pain Relief and Quality of Life Improvement in Dogs Affected by Spontaneous Osteoarthritis. Animals. 2020; 10:1505.
6. Brooks W. Phenobarbital. Veterinary Partner®0.2 Available at: https://veterinarypartner.vin.com/default.aspx?pid=19239&id=4951404
7. Clomicalm (clomipramine hydrochloride) Prescribing Information. Elanco Animal Health, Greenfield, IN.
8. Diess F. Understanding and treating noise aversion in dogs. Zoetis Petcare. 2023.
9. Engel O, Löscher W, Förster C. Effects of imepitoin on seizure threshold and hippocampal neurogenesis in a mouse model of epilepsy. Epilepsia Open. 2019; 4:368-380.
10. Garcia G A, Kube S, Carrera-Justiz S, Tittle D, Wakshlag J J. Safety and efficacy of cannabidiol-cannabidiolic acid rich hemp extract in the treatment of refractory epileptic seizures in dogs. Front Vet Sci. 2022; 9:939966.
11. Gertler R, Brown H C, Mitchell D H, Silvius E N. Dexmedetomidine: a novel sedative-analgesic agent. Proceedings (Baylor University Medical Center). 2001; 14 (1): 13-21.
12. Greb A, Puschner B. Cannabinoid treats as adjunctive therapy for pets: gaps in our knowledge. Toxicology Communications. 2018; 2 (1): 10-14.
13. Hurley E N, Ellaway C J, Johnson A M, Truong L, Gordon R, Galettis P, Martin J H, Lawson J A. Efficacy and safety of cannabidivarin treatment of epilepsy in girls with Rett syndrome: A phase 1 clinical trial. Epilepsia. 2022; 63:1736-1747.
14. Inkol J M, Hocker S E, Mutsaers A J. Combination therapy with cannabidiol and chemotherapeutics in canine urothelial carcinoma cells. PLOS ONE. 2021; 16 (8): e0255386.
15. Khodadadi H, Salles E L, Jarrahi A, Chibane F, Costigliola V, Yu J C, Vaibhav K, Khan M B, Hess D C, Dhandapani K M, Baban B. CBD reduces plaque, improves cognition in model of familial Alzheimer's. ScienceDaily. Mar. 9, 2021.
16. Lee J L C, Bertoglio L J, Guimarães F S, Stevenson C W. Cannabidiol regulation of emotion and emotional memory processing: relevance for treating anxiety-related and substance abuse disorders. British Journal of Pharmacology. 2017; 174:3242-3256.
17. McGrath S, Bartner L R, Rao S, Packer R A, Gustafson D L. Randomized blinded controlled clinical trial to assess the effect of oral cannabidiol administration in addition to conventional antiepileptic treatment on seizure frequency in dogs with intractable idiopathic epilepsy. JAVMA. 2019; 254:1301-1308.
18. Potschka H, Bhatti S F M, Tipold A, McGrath S. Cannabidiol in canine epilepsy. Vet Journal. 2022; 290:105913.
19. Reconcile (fluoxetine hydrochloride) Prescribing Information. Elanco Animal Health, Greenfield, IN.
20. Rozental A J, Weisbeck B G, Alvarenga I C, Gustafson D L, Kusick B R, Rao S, Bartner L R, McGrath S. The efficacy and safety of cannabidiol as adjunct treatment for drug-resistant idiopathic epilepsy in 51 dogs: A double-blinded crossover study. J Vet Intern Med. 2023; 37:2291-2300.
21. Sileo (dexmedetomidine oromucosal gel) Prescribing Information. Zoetis Inc., Kalamazoo, MI. Revised 2023.
22. Verrico C D, Wesson S, Konduri V, Hofferek C J, Vazquez-Perez J, Blair E, Dunner K Jr, Salazar L C, Alberts I, Sanchez R O, Neddenriep B, Jass J R, Eliav E, Costa B, Carmona R N, Gold M S. A randomized, double-blind, placebo-controlled study of daily cannabidiol for the treatment of canine osteoarthritis pain. Pain. 2020; 161 (9): 2191-2202.
23. Wang Z, Zheng H, Yang H, Song H, Lian J, Peng C, Wang H, Zhang H, Zheng Y, Wang Q, Lan L, Duan G, Ma L, Peng X, Huang Z. Cannabichromene from full-spectrum hemp extract exerts acute anti-seizure effects through allosteric activation of GABAA receptors. Fundamental Res. 2024; 4:1357-1364.

1. A veterinary pharmaceutical composition comprising:
;
;
nanometers;1 and

2. The composition of claim 1, wherein the CBD isolate has a purity of at least 99.9% of total cannabinoids.

3. The composition of claim 1, wherein the average particle size of the CBD isolate nano-particles is between 20 and 80 nanometers.

4. The composition of claim 1, wherein the composition contains less than 0.1% THC.

5. The composition of claim 1, wherein the oil-based medium is selected from the group consisting of medium-chain triglyceride (MCT) oil, hemp seed oil, olive oil, and combinations thereof.

6. The composition of claim 1, further comprising cannabigerol (CBG) isolate.

7. The composition of claim 6, wherein the ratio of CBD isolate to CBG isolate is between 1:1 and 9:1 by weight.

8. The composition of claim 1, further comprising cannabichromene (CBC) isolate.

9. A method for treating noise aversion in a non-human mammal, comprising:
;
wherein the CBD isolate has a purity of at least 99% of total cannabinoids and an average particle size of less than 100 nanometers; and
wherein the composition contains less than 0.1% THC;
wherein the composition is administered within 60 minutes prior to or during a noise event that induces anxiety in the non-human mammal.

10. The method of claim 9, wherein the non-human mammal is selected from the group consisting of dogs, cats, and horses.

11. The method of claim 9, wherein the composition is administered less than 20 minutes prior to the onset of the noise event.

12. The method of claim 9, wherein the effective amount is between 0.5 mg and 5 mg of CBD per kg of body weight of the non-human mammal.

13. The method of claim 9, wherein the noise event is selected from the group consisting of fireworks, thunderstorms, gunfire, and construction noise.

14. The method of claim 9, wherein the composition is administered orally or sublingually.

15. The method of claim 9, wherein administration of the composition is discontinued when the noise event subsides.

16. A veterinary pharmaceutical composition for treating seizures or idiopathic epilepsy in a non-human mammal, comprising:
cannabidiol (CBD) isolate;
cannabidivarin (CBDV) isolate; and
cannabichromene (CBC) isolate;
;
less than 100 nanometers; and

17. The composition of claim 16, wherein the CBD isolate, CBDV isolate, and CBC isolate each have a purity of at least 99% of total cannabinoids.

18. The composition of claim 16, wherein the CBD isolate, CBDV isolate, and CBC isolate are each in nano-particle form with an average particle size of less than 80 nanometers.

19. The composition of claim 16, wherein the cannabinoid isolates are suspended in an oil-based medium comprising medium-chain triglyceride (MCT) oil.

20. The composition of claim 16, wherein the non-human mammal is selected from the group consisting of dogs, cats, and horses.
